(19) United States
(12) Patent Application Publication
(10) Pub. No.: US 2021/0115493 A1
(43) Pub. Date: Apr. 22, 2021

(54) METHODS FOR DETERMINING A DRUG DOSING REGIMEN

(71) Applicant: Clear Creek Bio, Inc., Cambridge, MA (US)

(72) Inventors: David P. Hesson, Malvern, PA (US); Barbara Powers, Cambridge, MA (US); Vikram S. Kumar, Boston, MA (US)

(21) Appl. No.: 17/068,914

(22) Filed: Oct. 13, 2020

Related U.S. Application Data
(60) Provisional application No. 62/916,980, filed on Oct. 18, 2019.

(51) Int. Cl. C12Q 1/32; A61K 31/47

(52) U.S. Cl. CPC C12Q 1/32; A61K 31/47

(57) ABSTRACT

The invention provides methods for adjusting a drug dosing regimen, including a drug dosage and schedule of administration, in individuals who have previously received at least one dose of the drug. Adjustments to the dosage and/or schedule are made based on measured levels of the drug or metabolite of the drug and a biomarker of engagement of the drug with its target. The methods are useful for administration of therapeutic agents, such as brequinar, that alter metabolic pathways.

### CROSS-REFERENCE TO RELATED APPLICATIONS

This application claims the benefit of, and priority to, U.S. Provisional Patent Application No. 62/916,980, filed Oct. 18, 2019, the contents of which are incorporated by reference.

### FIELD OF THE INVENTION

The invention relates generally to methods of determining a drug dosing regimen for treatment of disease, disorder, or condition.

### BACKGROUND

Treatment of many serious medical conditions, such as cancers, entails repeated administration of potent therapeutic agents. Proper administration of such therapeutics is essential for the treatment to be effective: providing the agent at insufficient dosage and/or frequency fails to ameliorate the condition, while erring on the side of excessive dosage and/or frequency can lead to deleterious side effects.

Existing methods of determining drug dosing regimens are crude and inexact. Starting regimens are typically based on a patient's weight, age, and the type and severity of the condition. However, individual patients differ vastly in how they metabolize certain drugs, and those differences may be critical for drugs that have a narrow therapeutic window, i.e., a restricted range between the minimum dosage at which a drug is effective and the dosage at which it is toxic. Dosing regimens may be adjusted based on measured levels of the therapeutic agent or a metabolite of the agent in the patient's body, but such measurements do not necessarily reflect whether a particular agent has engaged its intended target. Consequently, current methods for optimizing drug dosing regimens are inadequate, and millions of individuals continue to suffer from improperly medicated ailments or unnecessary drug side effects.

### SUMMARY

The invention provides methods that allow physicians to adjust a drug dosing regimen in real time based on specific measurements of the pharmacokinetic and pharmacodynamic properties of the drug in a patient's body. The methods entail the use of measurements of both the therapeutic agent or a metabolite of the agent and a biomarker of engagement of the agent with its target in the body. The level of the agent or metabolite thereof serves as indicator of the patient's ability to metabolize the drug, while the level of the biomarker reveals the drug's efficacy at altering a targeted pathway. Because two independent parameters are used in determining whether to modify a dosing regimen, the methods permit adjustment of the dosage and frequency of administration with greater precision and accuracy. Moreover, the methods allow such adjustments following a single administration of the drug. Consequently, the methods of the invention enable physicians to make real-time modifications of a dosing regimen based on a patient-specific response to a drug to achieve optimal therapeutic benefit with minimal side effects.

Methods of the invention are useful in the treatment of diseases, such as cancer, that can be treated with agents that interfere with metabolic pathways. In particular, agents that interfere with nucleotide synthesis pathways are useful for treating cancer due to the high demand for nucleotides by actively proliferating tumor cells. The methods may be used with agents that target pyrimidine synthesis, purine synthesis, or both.

A useful therapeutic target involved in pyrimidine synthesis is dihydroorotate dehydrogenase (DHODH). The DHODH inhibitor brequinar can be used to treat acute myeloid leukemia (AML) due to its ability to selectively starve leukemia cells. However, dosing of brequinar must be carefully monitored to provide sustained inhibition of DHODH interrupted by brief periods that allow sufficient DHODH activity for healthy cells to survive. The methods of the invention allow fine-tuning of a brequinar dosing regimen based on measured levels brequinar and the DHODH substrate dihydroorotate (DHO) in the patient's blood. Consequently, after administration of one or more initial doses of brequinar to a patient, a physician has sufficient information to adjust the dosage and/or frequency of brequinar administration to obtain the optimal therapeutic outcome for that particular patient.

A potential therapeutic target required for purine synthesis is inosine monophosphate dehydrogenase (IMPDH). IMPDH can be blocked by tiazofurin, and several other IMPDH inhibitors are known. However, as with inhibition of DHODH, IMPDH inhibition must be monitored to ensure that cancer cells are being starved but healthy cells are not being harmed. The metabolites GTP and hypoxanthine can serve as biomarkers of IMPDH inhibition. Thus, by analyzing levels of both a therapeutic agent, such as tiazofurin, and a biomarker, such as GTP or hypoxanthine, methods of the invention allow careful adjustment of dosing regimens for IMPDH inhibitors as well.

In an aspect, the invention provides methods for adjusting a dose and/or schedule of dosing of an agent provided to a subject that has a disorder. The methods include receiving a sample from a subject that has received an initial dose of an agent; conducting a first assay on the sample to assess whether a level of the agent in the sample is within a therapeutic window; conducting a second assay on the sample that assesses a biomarker that is indicative of whether the agent has engaged a biological target, wherein the assay assesses whether the biomarker is below or above a threshold level; and generating a report that provides (i) a level of the agent in the sample with respect to the therapeutic window and (ii) a level of the biomarker with respect to the threshold level, wherein the report provides guidance to a doctor on how to determine a dosage adjustment for a subsequent dose of the agent and/or an adjustment of a schedule of dosing of the agent to be provided to the subject based on (i) and (ii) of the report.

The sample may be blood sample, plasma sample, serum sample, or urine sample.

The method may include an adjustment to the dosage, an adjustment to the dosing schedule, or both. The dosage adjustment may be an increase the dosage by a certain amount, a decrease the dosage by a certain amount, and no adjustment to the dosage. The adjustment of the schedule of dosing may be an increase in frequency in which the agent is provided, a decrease the frequency in which the agent is provided, or no adjustment to the frequency.

The agent may inhibit an enzyme in a metabolic pathway. The metabolic pathway may be a nucleotide synthesis pathway. The enzyme may be transcarbamoylase, dihydrooratase, dihydroorotate dehydrogenase, orotidine 5′-monophosphate (OMP) decarboxylase, inositol monophosphate dehydrogenase, or orotate phosphoribosyl transferase.

The metabolite may be a substrate of the enzyme. The metabolite may be N-carbamoylaspartate, dihydroorotate, orotate, orotidine 5′-monophosphate (OMP), inosine monophosphate (IMP), hypoxanthine, guanosine triphosphate (GTP), uridine, or uridine monophosphate (UMP).

The agent may be PALA (N-phosphoacetyl-L-aspartate), pyrazofurin, brequinar, a brequinar analog, a brequinar derivative, a brequinar prodrug, a micellular formulation of brequinar, a brequinar salt, mizoribine, mycophenolic acid, ribavirin, selenazofurin, taribavirin, or tiazofurin.

The disorder may be any disease, disorder, or condition for which a DHODH inhibitor would provide a therapeutic benefit. The disorder may be cancer. The cancer may be leukemia (e.g., acute myeloid leukemia) or prostate cancer. The disorder may be an autoimmune disease, such as multiple sclerosis or arthritis (e.g., rheumatoid arthritis or psoriatic arthritis).

The method may include providing the agent to the subject at the adjusted dosage, adjusted schedule, or both.

In another aspect, the invention provides methods for adjusting a dose and/or dosing schedule of an agent to be provided to a subject that has a disorder and that has already received an initial dose of the agent. The methods include receiving, after a subject has received an initial dose of an agent, a report that provides (i) a level of the agent in a sample with respect to a therapeutic window and (ii) a level of a biomarker in a sample with respect to a threshold level, wherein the biomarker is indicative of whether an agent has engaged a biological target; determining, based on (i) and (ii) in the report, a dosage adjustment for a subsequent dose of the agent to be provided to the subject and/or an adjustment of a schedule of dosing of the agent to be provided to the subject; and providing the agent to the subject at the determined adjusted dose and/or adjusted dosing schedule.

The sample may be blood sample, plasma sample, serum sample, or urine sample.

The method may include an adjustment to the dosage, an adjustment to the dosing schedule, or both, as described above.

The agent may inhibit an enzyme in a metabolic pathway, as described above. The agent may be any agent described above.

The metabolite may be a substrate of the enzyme, as described above. The metabolite may be any metabolite described above.

The disorder may be any disorder described above.

In another aspect, the invention provides methods for determining whether a subject receiving once-a-week dosing of brequinar should receive twice-a-week dosing of brequinar. The methods include determining whether the following conditions are met in a subject that is being provided brequinar on a once-a-week dosing schedule: (i) whether a plurality of plasma levels of brequinar in the subject are within a therapeutic window; (ii) whether a level of dihydroorotate dehydrogenase (DHO) in the subject at a second time point is less than a level of DHO in the subject at a first time point, and (iii) whether the level of DHO in the subject at the second time point is less than a DHO threshold level. The methods further include providing brequinar to the subject on a twice-a-week dosing schedule if (i), (ii), and (iii) are satisfied.

The first time point may be about 6 hours after the subject has received the dose of brequinar, about 12 hours after the subject has received the dose of brequinar, about 18 hours after the subject has received the dose of brequinar, about 24 hours after the subject has received the dose of brequinar, about 36 hours after the subject has received the dose of brequinar, or about 48 hours after the subject has received the dose of brequinar.

The second time point may be about 24 hours after the subject has received the dose of brequinar, about 36 hours after the subject has received the dose of brequinar, about 48 hours after the subject has received the dose of brequinar, about 60 hours after the subject has received the dose of brequinar, about 72 hours after the subject has received the dose of brequinar, or about 84 hours after the subject has received the dose of brequinar.

The therapeutic window may include a first plasma level of brequinar greater than or equal to a plasma threshold level at 24 hours after the subject has received the dose of brequinar and a second plasma level of brequinar less than and/or equal to the plasma threshold level at 48 hours after the subject has received the dose of brequinar. The plasma threshold level may be about 1 μg/mL, about 2 μg/mL, about 3 μg/mL, about 4 μg/mL, about 5 μg/mL, about 6 μg/mL, about 8 μg/mL, or about 10 μg/mL.

The method may include increasing the amount of brequinar in a dose if the first plasma level at the first time point is less than a defined value. The defined value may be about 1 μg/mL, about 2 μg/mL, about 3 μg/mL, about 4 μg/mL, about 5 μg/mL, about 6 μg/mL, about 8 μg/mL, or about 10 μg/mL.

The amount of brequinar in a dose may be increased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be increased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m².

The method may include continuing the once-a-week dosing schedule using the dose with the increased amount of brequinar if the first plasma level at the first time point is less than the defined value.

The method may include continuing the method if the first plasma level at the first time point is greater than or equal to a defined value and determining the second plasma level at the second time point.

The method may include decreasing the amount of brequinar in a dose if the second plasma level at the second time point is greater than a defined value. The defined value may be about 1 μg/mL, about 2 μg/mL, about 3 μg/mL, about 4 μg/mL, about 5 μg/mL, about 6 μg/mL, about 8 μg/mL, or about 10 μg/mL.

The dose of brequinar may be decreased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The dose of brequinar may be decreased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m². The dose may be decreased to contain an amount of brequinar that is not less than a previously-used amount.

The method may include continuing the once-a-week dosing schedule using the dose with the decreased amount of brequinar if the second plasma level at the second time point is less than the defined value.

The method may include obtaining one or more DHO levels at defined time points if the first and second plasma levels of brequinar are within the therapeutic window. The defined time point may be about 6 hours after the subject has received the dose of brequinar, about 12 hours after the subject has received the dose of brequinar, about 18 hours after the subject has received the dose of brequinar, about 24 hours after the subject has received the dose of brequinar, about 36 hours after the subject has received the dose of brequinar, about 48 hours after the subject has received the dose of brequinar, about 60 hours after the subject has received the dose of brequinar, about 72 hours after the subject has received the dose of brequinar, or about 84 hours after the subject has received the dose of brequinar.

The method may include providing brequinar on a twice-a-week dosing schedule if the DHO level at the second time point is less than the DHO level at the first time point, less than a DHO threshold level, or both. The first and second time points may be 48 and 72 hours, respectively. The DHO threshold level may be about 1000 ng/mL, about 2000 ng/mL, about 3000 ng/mL, about 4000 ng/mL, about 5000 ng/mL, about 6000 ng/mL, about 8000 ng/mL, or about 10,000 ng/mL.

The method may include continuing the once-a-week dosing schedule using the decreased dose of brequinar if the DHO level at the second time is greater than or equal to the DHO level at the first time point, greater than or equal to a DHO threshold level, or both. The first and second time points may be 48 and 72 hours, respectively. The DHO threshold level may be about 1000 ng/mL, about 2000 ng/mL, about 3000 ng/mL, about 4000 ng/mL, about 5000 ng/mL, about 6000 ng/mL, about 8000 ng/mL, or about 10,000 ng/mL.

The once-a-week dosing schedule may be continued using a dose containing a decreased amount of brequinar. The dose of brequinar may be decreased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The dose of brequinar may be decreased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m². The dose of brequinar may be decreased to an amount that is not less than a previously used amount.

The initial dose of brequinar may be about 200 mg/m², about 300 mg/m², about 400 mg/m², about 500 mg/m², about 600 mg/m², about 700 mg/m², about 800 mg/m², from about 200 mg/m² to about 800 mg/m², from about 200 mg/m² to about 700 mg/m², from about 200 mg/m² to about 600 mg/m², from about 200 mg/m² to about 500 mg/m², from about 300 mg/m² to about 800 mg/m², from about 300 mg/m² to about 700 mg/m², from about 300 mg/m² to about 600 mg/m², from about 300 mg/m² to about 500 mg/m², from about 400 mg/m² to about 800 mg/m², from about 400 mg/m² to about 700 mg/m², from about 400 mg/m² to about 600 mg/m², from about 400 mg/m² to about 500 mg/m², from about 500 mg/m² to about 800 mg/m², from about 500 mg/m² to about 700 mg/m², or from about 500 mg/m² to about 600 mg/m².

In another aspect, the invention provides methods of assessing a twice-a-week dosing schedule of brequinar that is being provided to a subject. The methods include determining in a subject that is on a twice-a-week dosing schedule of brequinar whether a dihydroorotate dehydrogenase (DHO) level is within a therapeutic window at a defined time point after the subject has received a dose containing a first amount of brequinar and continuing to provide a dose that contains a greater or equal amount of brequinar to the subject on the twice-a-week dosing schedule if the DHO level is within the therapeutic window.

The dose in the continued twice-a-week dosing schedule may contain the same amount of brequinar as the first amount. The dose in the continued twice-a-week dosing schedule may contain an amount that is greater than the first amount.

The defined time point may be about 60 hours after the subject has received a dose of brequinar, about 72 hours after the subject has received a dose of brequinar, about 84 hours after the subject has received a dose of brequinar, about 96 hours after the subject has received a dose of brequinar, or about 108 hours after the subject has received a dose of brequinar.

The determining step may be performed repeatedly at a defined interval for a defined period. The determining step may be performed three times per week, two times per week, once per week, or biweekly. The determining step may be performed repeatedly for 4 weeks, 6 weeks, 8 weeks, 10 weeks, 12 weeks, 16 weeks, 20 weeks, or 24 weeks. The determining step may be performed weekly for a first twelve weeks of the twice-a-week dosing schedule. The determining step may be performed every two weeks after the first twelve weeks.

The therapeutic window may be DHO level greater than or equal to about 500 ng/mL and less than about 3000 ng/mL, greater than or equal to about 1000 ng/mL and less than about 3000 ng/mL, greater than or equal to about 1500 ng/mL and less than about 3000 ng/mL, greater than or equal to about 2000 ng/mL and less than about 3000 ng/mL, greater than or equal to about 500 ng/mL and less than about 4000 ng/mL, greater than or equal to about 1000 ng/mL and less than about 4000 ng/mL, greater than or equal to about 1500 ng/mL and less than about 4000 ng/mL, greater than or equal to about 2000 ng/mL and less than about 4000 ng/mL, greater than or equal to about 2500 ng/mL and less than about 4000 ng/mL, greater than or equal to about 500 ng/mL and less than about 5000 ng/mL, greater than or equal to about 1000 ng/mL and less than about 5000 ng/mL, greater than or equal to about 1500 ng/mL and less than about 5000 ng/mL, greater than or equal to about 2000 ng/mL and less than about 5000 ng/mL, greater than or equal to about 2500 ng/mL and less than about 5000 ng/mL, greater than or equal to about 500 ng/mL and less than about 6000 ng/mL, greater than or equal to about 1000 ng/mL and less than about 6000 ng/mL, greater than or equal to about 1500 ng/mL and less than about 6000 ng/mL, greater than or equal to about 2000 ng/mL and less than about 6000 ng/mL, greater than or equal to about 2500 ng/mL and less than about 6000 ng/mL, greater than or equal to about 500 ng/mL and less than about 7500 ng/mL, greater than or equal to about 1000 ng/mL and less than about 7500 ng/mL, greater than or equal to about 1500 ng/mL and less than about 7500 ng/mL, greater than or equal to about 2000 ng/mL and less than about 7500 ng/mL, or greater than or equal to about 2500 ng/mL and less than about 7500 ng/mL.

The method may include increasing the amount of brequinar in a dose and continuing the twice-a-week dosing schedule using the increased amount of brequinar if the DHO level at the defined time point is less than a DHO threshold level. The DHO threshold level may be about 500 ng/mL, about 750 ng/mL, about 1000 ng/mL, about 1250 ng/mL, about 1500 ng/mL, about 2000 ng/mL, or about 2500 ng/mL.

The amount of brequinar in a dose may be increased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be increased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m².

The method may include not giving the subject, i.e., withholding from the subject, brequinar for a period if the DHO level is greater than or equal to a threshold level. The threshold level may be about 2000 ng/mL, about 3000 ng/mL, about 4000 ng/mL, about 5000 ng/mL, about 6000 ng/mL, about 8000 ng/mL, or about 10,000 ng/mL. The period may be about 3.5 days, about one week, about 10.5 days, or about two weeks. The period may be defined by the time point when the subject was given a dose of brequinar or by a subsequent time point, such as when the DHO level was determined.

The method may include resuming a twice-a-week dosing schedule using a dose containing a decreased amount of brequinar following a dosing-free period, i.e., a period in which is the subject is not given brequinar. The amount of brequinar in a dose may be decreased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be decreased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m². The dose may be decreased to contain an amount of brequinar that is not less than a previously-used amount.

The method may include determining whether the subject suffers from an unsafe adverse event. The method may include continuing to provide brequinar on a twice-a-week dosing schedule if the subject does not suffer from an unsafe adverse event. The unsafe adverse event may be or include a ≥grade 3, treatment-emergent, study-drug-related, non-hematologic adverse event.

### DETAILED DESCRIPTION

The invention provides improved methods for making adjustments to a drug dosing regimen. The methods allow adjustment of the drug dosage and/or schedule of administration following an initial administration of one or more doses. The methods rely on two independent parameters to determine whether a dosing regimen should be modified. First, levels of therapeutic agent or a metabolite of the agent in the blood are used as an indicator of an individual patient's ability to metabolize the drug. The methods also use levels of a biomarker that indicates the extent to which the agent has engaged its biological target. By incorporating both pharmacokinetic and pharmacodynamics measurements, the methods allow fine-tuning of drug dosing regimens to achieve optimal therapeutic benefit with minimal side effects.

Methods of the invention overcome the difficulty of making a priori predictions of how an individual patient will respond to a particular drug. Initial drug dosing regimens are typically based on a comparison of basic features of an individual patient, such as age, weight, sex, and the type and status of her condition, to prior patients with similar features. However, seemingly similar patients may display high variability in their responses to the same drug dosing regimen.

As a result, when a physician chooses a drug dosing regimen and follows the chosen course of therapy for a given individual, success of treatment is largely dependent on fortuitous elements. The methods provided herein relieve physicians from being beholden to their own guesswork by allowing them to change course based on empirical data on how a given patient responds to a drug.

### Determining Whether to Adjust a Dosing Regimen

As used herein, a “dosing regimen” for a drug may include a dosage and a schedule of administration. Thus, adjustments to a drug dosing regimen may include modifications to the dosage, schedule of administration, or both. A dosage may be described by an absolute amount of drug, e.g., a mass or concentration times volume, or by a relative amount of the drug to the subject, e.g., mass of drug per mass of subject or mass of drug per volume of subject. A schedule of administration may be described by the interval between doses, e.g., every 24 hours, every 48 hours, etc., or by the number doses administered during a given period, e.g., once per week, twice per week, etc.

Methods of the invention include the use of measurements of two parameters from one or more samples that have been obtained from a subject, e.g., a patient, that has previously received one or more doses of a drug: (1) the level therapeutic agent or a metabolite of the agent and (2) the level of a biomarker of engagement of the agent with its target. Any suitable therapeutic agent may be used in methods of the invention, and examples are described in detail below. The biomarker may be a metabolite in a pathway of an enzyme that is targeted by the agent or therapeutic agent, and examples are described in detail below.

Both the therapeutic agent and the biomarker may have therapeutic windows in which the optimal benefit is obtained by the subject. The therapeutic window may be a range of concentrations in which the therapeutic agent is altering the activity of its target to achieve a beneficial effect and but is having minimal direct or collateral effects that are harmful to the subject. A therapeutic window may be bounded at both the upper and lower ends to form a closed range. Alternatively, a therapeutic window may be bounded only at the upper end or only at the lower end to form an open range.

The therapeutic window of a therapeutic agent or biomarker may be defined at a given time point relative to administration of the therapeutic agent to the subject. For example and without limitation, the therapeutic window may be defined at 24 hours post-administration, 36 hours post-administration, 48 hours post-administration, 60 hours post-administration, 72 hours post-administration, 84 hours post-administration, 96 hours post-administration, 108 hours post-administration, or 120 hours post-administration. The therapeutic window of a therapeutic agent and the therapeutic window of a biomarker may be defined at the same time point, or they may be defined at different time points.

Determinations of whether to adjust a dosing regimen may include measurement of the levels of a therapeutic agent or a metabolite thereof and a biomarker in a sample obtained from a subject. Alternatively or additionally, determinations of whether to adjust a dosing regimen may include receiving information provided in a report on the levels of the therapeutic agent or metabolite thereof and biomarker in a sample obtained from a subject. Determinations of whether to adjust a dosing regimen may include providing a therapeutic agent to a subject according to an adjusted dosing regimen. Alternatively or additionally, determinations of whether to adjust a dosing regimen may include providing a report with instructions or recommendations on how to provide a therapeutic agent to a subject according to an adjusted dosing regimen.

The adjustment to the dosing regimen may include a change in the dosage. For example, the adjustment to the dosing regimen may include an increase in the dosage or a decrease in the dosage. The adjustment to the dosing regimen may include no change to the dosage.

The adjustment to the dosing regimen may include a change in the schedule of administration. For example, adjustment to the dosing regimen may include an increase in the frequency of administration or a decrease in the frequency of administration. The adjustment to the dosing regimen may include no change in the schedule of administration.

The adjustment to the dosing regimen may include refraining from administration of one or more scheduled doses, i.e., withholding from the subject one or more doses. The adjustment to the dosing regimen may include refraining from administration of one or more scheduled doses for a defined period. The defined period may be 1 day, 2 days, 3 days, 4 day, 5 days, 6 days, 7 days, half a week, one week, or two weeks.

The determination of whether a dosing regimen should be adjusted may include determining whether one or more conditions are met. For example, the determination of whether a dosing regimen should be adjusted may include determining whether one or more of the following conditions are met: whether the level of the therapeutic agent or metabolite thereof in one or more samples obtained from a subject are within a therapeutic window; whether the level of a biomarker in a sample obtained from a subject at one time point is greater than, less than, or equal to the level of a biomarker in a sample obtained from a subject at another time point; and whether the level of biomarker in a sample obtained from a subject at a time point is greater than, less than, or equal to a threshold.

Examples of specific embodiments of the invention are provided below.

The method may include determining whether a subject receiving once-a-week dosing of brequinar should receive twice-a-week dosing of brequinar.

The method may include determining whether the following conditions are met in a subject that is being provided brequinar on a once-a-week dosing schedule: (i) whether a plurality of plasma levels of brequinar in the subject are within a therapeutic window; (ii) whether a level of dihydroorotate dehydrogenase (DHO) in the subject at a second time point is less than a level of DHO in the subject at a first time point, and (iii) whether the level of DHO in the subject at the second time point is less than a DHO threshold level.

The method may include providing brequinar to the subject on a twice-a-week dosing schedule if each of conditions (i), (ii), and (iii) is satisfied.

The first time point may be about 6 hours after the subject has received the dose of brequinar, about 12 hours after the subject has received the dose of brequinar, about 18 hours after the subject has received the dose of brequinar, about 24 hours after the subject has received the dose of brequinar, about 36 hours after the subject has received the dose of brequinar, or about 48 hours after the subject has received the dose of brequinar.

The second time point may be about 24 hours after the subject has received the dose of brequinar, about 36 hours after the subject has received the dose of brequinar, about 48 hours after the subject has received the dose of brequinar, about 60 hours after the subject has received the dose of brequinar, about 72 hours after the subject has received the dose of brequinar, or about 84 hours after the subject has received the dose of brequinar.

The therapeutic window may include a first plasma level of brequinar greater than or equal to a plasma threshold level at 24 hours after the subject has received the dose of brequinar and a second plasma level of brequinar less than or equal to the plasma threshold level at 48 hours after the subject has received the dose of brequinar. The plasma threshold level may be about 1 μg/mL, about 2 μg/mL, about 3 μg/mL, about 4 μg/mL, about 5 μg/mL, about 6 μg/mL, about 8 μg/mL, or about 10 μg/mL.

The method may include increasing the dose of brequinar if the first plasma level at the first time point is less than or equal to a defined value. The defined value may be about 1 μg/mL, about 2 μg/mL, about 3 μg/mL, about 4 μg/mL, about 5 μg/mL, about 6 μg/mL, about 8 μg/mL, or about 10 μg/mL.

The amount of brequinar in a dose may be increased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be increased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m².

The method may include continuing the once-a-week dosing schedule using the dose with the increased amount of brequinar if the first plasma level at the first time point is less than or equal to the defined value.

The method may include continuing the method if the first plasma level at the first time point is greater than or equal to a defined value and determining the second plasma level at the second time point.

The method may include decreasing the dose of brequinar if the second plasma level at the second time point is greater than a defined value. The defined value may be about 1 μg/mL, about 2 μg/mL, about 3 μg/mL, about 4 μg/mL, about 5 μg/mL, about 6 μg/mL, about 8 μg/mL, or about 10 μg/mL.

The amount of brequinar in a dose may be decreased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be decreased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m².

Because methods of the invention contemplate both increases and decreases in the dosed amount of brequinar in response to levels of a therapeutic agent, metabolite, or both in samples from the subject, it may be necessary to limit the scope of adjustments to dosing amounts. This can be achieved by requiring that the adjusted amount in a second or later adjustment not exceed or drop below a previously-used amount. For example, a method may begin with a first dosing amount, require an upward adjustment to a second dosing amount, and then require a downward adjustment to a third dosing amount. In such instances, the method may require that the third dosing amount not be less than the first dosing amount, thereby providing a lower limit for the range of the second adjustment. Thus, the dose may be decreased to contain an amount of brequinar that is not less than a previously-used amount. Conversely, a method may begin with a first dosing amount, require a downward adjustment to a second dosing amount, and then require an upward adjustment to a third dosing amount. In such instances, the method may require that the third dosing amount not be greater than the first dosing amount, thereby providing an upper limit for the range of the second adjustment. Thus, the dose may be increased to contain an amount of brequinar that is not greater than a previously-used amount.

The method may include continuing the once-a-week dosing schedule using the dose with a decreased amount of brequinar if the second plasma level at the second time point is less than or equal to the defined value.

The method may include obtaining one or more DHO levels at defined time points if the first and second plasma levels of brequinar are within the therapeutic window. A DHO level may be obtained at a first time point of about 6 hours after the subject has received the dose of brequinar, about 12 hours after the subject has received the dose of brequinar, about 18 hours after the subject has received the dose of brequinar, about 24 hours after the subject has received the dose of brequinar, about 36 hours after the subject has received the dose of brequinar, about 48 hours after the subject has received the dose of brequinar, about 60 hours after the subject has received the dose of brequinar, or about 72 hours after the subject has received the dose of brequinar. A DHO level may be obtained at a second time point of about 24 hours after the subject has received the dose of brequinar, about 36 hours after the subject has received the dose of brequinar, about 48 hours after the subject has received the dose of brequinar, about 60 hours after the subject has received the dose of brequinar, about 72 hours after the subject has received the dose of brequinar, about 84 hours after the subject has received the dose of brequinar, or about 96 hours after the subject has received the dose of brequinar.

The method may include providing brequinar on a twice-a-week dosing schedule if the DHO level at 72 hours is less than the DHO level at 48 hours, less than a DHO threshold level, or both. The DHO threshold level may be about 1000 ng/mL, about 2000 ng/mL, about 3000 ng/mL, about 4000 ng/mL, about 5000 ng/mL, about 6000 ng/mL, about 8000 ng/mL, or about 10,000 ng/mL.

The method may include decreasing the brequinar dose and continuing the once-a-week dosing schedule using the decreased dose of brequinar if the DHO level at 72 hours is less than the DHO level at 48 hours, less than a DHO threshold level, or both. The amount of brequinar in a dose may be decreased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be decreased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m². The DHO threshold level may be about 1000 ng/mL, about 2000 ng/mL, about 3000 ng/mL, about 4000 ng/mL, about 5000 ng/mL, about 6000 ng/mL, about 8000 ng/mL, or about 10,000 ng/mL.

The initial dose of brequinar may be about 200 mg/m², about 300 mg/m², about 400 mg/m², about 500 mg/m², about 600 mg/m², about 700 mg/m², or about 800 mg/m².

The method may include assessing a twice-a-week dosing schedule of brequinar that is being provided to a subject.

The method may include determining in a subject that is on a twice-a-week dosing schedule of brequinar whether a dihydroorotate dehydrogenase (DHO) level is within a therapeutic window at a defined time point after the subject has received a dose of brequinar and continuing to provide a same dose of brequinar to the subject on the twice-a-week dosing schedule if the DHO level is within the therapeutic window.

The defined time point may be about 60 hours after the subject has received a dose of brequinar, about 72 hours after the subject has received a dose of brequinar, about 84 hours after the subject has received a dose of brequinar, about 96 hours after the subject has received a dose of brequinar, or about 108 hours after the subject has received a dose of brequinar.

The determining step may be performed repeatedly at a defined interval for a defined period. The determining step may be performed three times per week, two times per week, once per week, or biweekly. The determining step may be performed repeatedly for 4 weeks, 6 weeks, 8 weeks, 10 weeks, 12 weeks, 16 weeks, 20 weeks, or 24 weeks. The determining step may be performed weekly for a first twelve weeks of the twice-a-week dosing schedule. The determining step may be performed every two weeks after the first twelve weeks.

The therapeutic window may be DHO level greater than or equal to about 500 ng/mL and less than about 3000 ng/mL, greater than or equal to about 1000 ng/mL and less than about 3000 ng/mL, greater than or equal to about 1500 ng/mL and less than about 3000 ng/mL, greater than or equal to about 2000 ng/mL and less than about 3000 ng/mL, greater than or equal to about 500 ng/mL and less than about 4000 ng/mL, greater than or equal to about 1000 ng/mL and less than about 4000 ng/mL, greater than or equal to about 1500 ng/mL and less than about 4000 ng/mL, greater than or equal to about 2000 ng/mL and less than about 4000 ng/mL, greater than or equal to about 2500 ng/mL and less than about 4000 ng/mL, greater than or equal to about 500 ng/mL and less than about 5000 ng/mL, greater than or equal to about 1000 ng/mL and less than about 5000 ng/mL, greater than or equal to about 1500 ng/mL and less than about 5000 ng/mL, greater than or equal to about 2000 ng/mL and less than about 5000 ng/mL, greater than or equal to about 2500 ng/mL and less than about 5000 ng/mL, greater than or equal to about 500 ng/mL and less than about 6000 ng/mL, greater than or equal to about 1000 ng/mL and less than about 6000 ng/mL, greater than or equal to about 1500 ng/mL and less than about 6000 ng/mL, greater than or equal to about 2000 ng/mL and less than about 6000 ng/mL, greater than or equal to about 2500 ng/mL and less than about 6000 ng/mL, greater than or equal to about 500 ng/mL and less than about 7500 ng/mL, greater than or equal to about 1000 ng/mL and less than about 7500 ng/mL, greater than or equal to about 1500 ng/mL and less than about 7500 ng/mL, greater than or equal to about 2000 ng/mL and less than about 7500 ng/mL, or greater than or equal to about 2500 ng/mL and less than about 7500 ng/mL.

The method may include increasing the brequinar dose and continuing the twice-a-week dosing schedule using the increased dose of brequinar if the DHO level at the defined time point is less than a DHO threshold level. The amount of brequinar in a dose may be increased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be increased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m². The DHO threshold level may be about 500 ng/mL, about 750 ng/mL, about 1000 ng/mL, about 1250 ng/mL, about 1500 ng/mL, about 2000 ng/mL, or about 2500 ng/mL.

The method may include having the subject not take the next dose of brequinar if the DHO level at the defined time point is greater than or equal to a threshold value. The threshold level may be about 2000 ng/mL, about 3000 ng/mL, about 4000 ng/mL, about 5000 ng/mL, about 6000 ng/mL, about 8000 ng/mL, or about 10,000 ng/mL. The method may include notifying the subject not to take the next dose of brequinar if the DHO level at the defined time point is greater than or equal to a threshold value. The threshold level may be about 2000 ng/mL, about 3000 ng/mL, about 4000 ng/mL, about 5000 ng/mL, about 6000 ng/mL, about 8000 ng/mL, or about 10,000 ng/mL. The method may include a dosing-free period if the DHO level at the defined time point is greater than or equal to a threshold value. The dosing-free period may be about 3.5 days, one week, 10.5 days, or two weeks.

The method may include resuming a twice-a-week dosing schedule using a decreased dose of brequinar following a dosing-free period if the DHO level at the defined time point is greater than or equal to a threshold value. The method may include notifying the subject to resume a twice-a-week dosing schedule using a decreased dose of brequinar following a dosing-free period if the DHO level at the defined time point is greater than or equal to a threshold value.

The amount of brequinar in a dose may be increased by about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², or about 500 mg/m². The amount of brequinar in a dose may be increased by from about 25 mg/m² to about 500 mg/m², from about 50 mg/m² to about 500 mg/m², from about 75 mg/m² to about 500 mg/m², from about 100 mg/m² to about 500 mg/m², from about 25 mg/m² to about 400 mg/m², from about 50 mg/m² to about 400 mg/m², from about 75 mg/m² to about 400 mg/m², from about 100 mg/m² to about 400 mg/m², from about 25 mg/m² to about 300 mg/m², from about 50 mg/m² to about 300 mg/m², from about 75 mg/m² to about 300 mg/m², from about 100 mg/m² to about 300 mg/m², from about 25 mg/m² to about 200 mg/m², from about 50 mg/m² to about 200 mg/m², from about 75 mg/m² to about 200 mg/m², or from about 100 mg/m² to about 200 mg/m².

### Metabolites as Biomarkers of Target Engagement

Methods of the invention include the use of measured levels of a biomarker of engagement of a target by a therapeutic agent to determine whether to make adjustments to a dosing regimen. The biomarker may be any molecule that provides an indication of target engagement by the therapeutic agent. In embodiments of the invention, the therapeutic agent is an inhibitor of an enzyme in a metabolic pathway, and the metabolite is an intermediate the pathway. Preferably, the agent is an inhibitor of an enzyme in a metabolic pathway, and the metabolite is a substrate of the enzyme.

Nucleotide synthesis pathways are of particular therapeutic interest. The high proliferation rate of cancer cells often places increased demand on nucleotide synthesis pathways. Consequently, enzymes that function in such pathways are useful targets for antineoplastic drugs. Specifically, drugs that inhibit enzymes require for nucleotide synthesis have been investigated for treating cancer. Therefore, levels of metabolites in nucleotide synthesis pathways are useful for evaluating the extent to which the therapeutic agents in such drugs are engaging their targets in vivo.

Pyrimidine biosynthesis involves a sequence of step enzymatic reactions that result in the conversion of glutamine to uridine monophosphate as shown below:

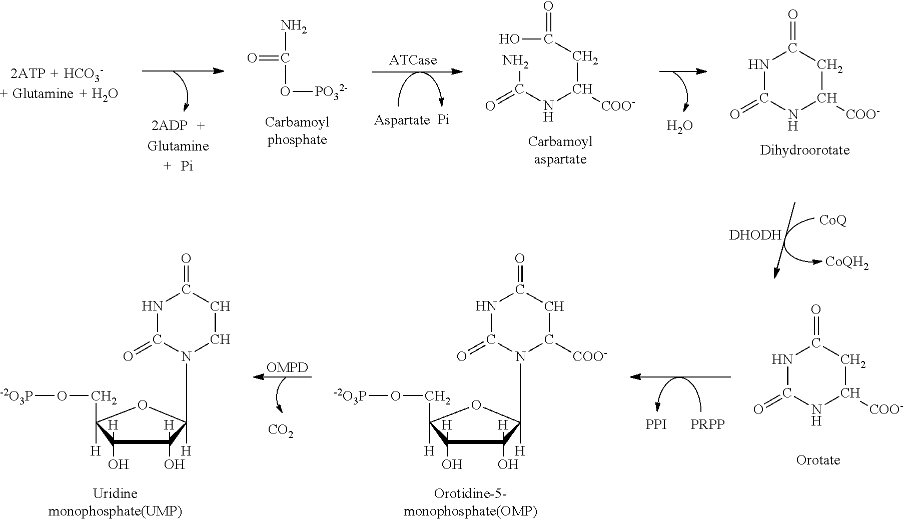

Several of the enzymes in the pyridine synthesis pathway are targets of drugs or drug candidates. For example, inhibitors of the following enzymes have been investigated as therapeutic agents: aspartate carbamoyltransferase (also known as aspartate transcarbamoylase or ATCase), which catalyzes the conversion of carbamoyl phosphate to carbamoyl aspartate; dihydroorotate dehydrogenase (DHODH), which catalyzes conversion of dihydroorotate (DHO) to orotate; and OMP decarboxylase (OMPD), which catalyzes conversion of orotidine monophosphate (OMP) to uridine monophosphate (UMP).

DHO is a useful indicator of target engagement by DHODH inhibitors. One advantage of DHO is that cell membranes are permeable to the molecule. DHODH is localized to the mitochondrial inner membrane within cells, making direct measurement of enzyme activity difficult. However, DHO, which accumulates when DHODH is inhibited, diffuses out of cells and into the blood, which can be easily sampled. Moreover, DHO is sufficiently stable that levels of the metabolite can be measured reliably by methods provided herein. Thus, by analyzing levels of DHO in blood or blood products, one can readily assess target engagement of a DHODH inhibitor. The use of DHO as an indicator of engagement by DHODH inhibitors is described in, for example, co-pending, co-owned U.S. application Ser. Nos. 16/364,423; 16/364,431; 16/364,436; 16/364,446; 62/838730; 62/859319; and 62/851,291, the contents of each of which are incorporated herein by reference.

In an analogous manner, orotate and OMP can serve as indicators for target engagement of OMP decarboxylase inhibitors. For example, inhibition of OMP decarboxylase leads to increased plasma levels of orotate, so measurement of plasma orotate levels is useful for assessing the effect of agents that target OMP decarboxylase. The use of OMP as an indicator of target engagement by OMP decarboxylase inhibitors is described in, for example, co-pending, co-owned U.S. application Ser. Nos. 16/364,423; 16/364,431; 16/364,436; and Ser. No. 16/364,446, the contents of each of which are incorporated herein by reference.

Agents that inhibit purine synthesis are also useful in methods of the invention. As indicated above, cancer cells have high rates of DNA replication, and DNA replication requires an ample supply of purines as well as pyrimidines. Consequently, several enzymes involved in synthesis of purines, such as guanosine, are also of therapeutic interest. Some of the key reactions involved in synthesis of guanine nucleotides are shown below:

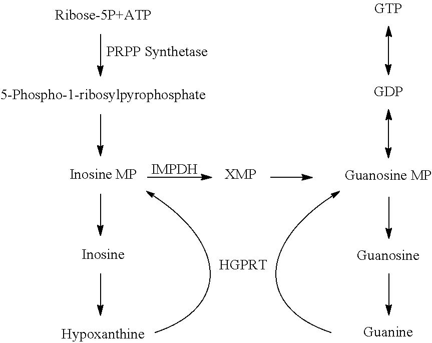

The multi-step conversion of ribose-5-phosphate (ribose-5P) to inosine monophosphate shown in the upper left portion of the diagram is required for all de novo purine synthesis. For synthesis of guanine nucleotides, such guanosine triphosphate (GTP) and deoxyguanosine triphosphate (dGTP), inosine monophosphate dehydrogenase (IMPDH) catalyzes the nicotinamide adenine dinucleotide (NAD⁺)-dependent oxidation of inosine monophosphate (IMP) to xanthosine monophosphate (XMP). Guanosine monophosphate synthetase (GMPS) then converts XMP to guanosine monophosphate. For synthesis of GTP, shown in the upper right portion of the diagram, two more phosphate moieties are added stepwise to create guanosine diphosphate (GDP) first and then GTP. For synthesis of dGTP, a building block for DNA replication, GDP is converted to deoxyguanosine diphosphate (dGDP), which is then phosphorylated to produce dGTP.

Purines can also be synthesized from the components of degraded macromolecules via salvage pathways. The lower half of the diagram shows that IMP and GMP can be broken down to hypoxanthine and guanine, respectively, via multiple steps. However, hypoxanthine-guanine phosphoribosyltransferase (HGPRT) reverses both series of reactions to regenerate IMP and GMP under appropriate conditions. Thus, HGPRT bypasses IMPDH to synthesize guanine nucleotides via the guanine salvage pathway. Certain tissues and organs are unable to undergo de novo synthesis of purines and rely exclusively on salvage pathways to supply needed nucleotides.

Hypoxanthine can be sequentially converted by xanthine oxidase (XO) first to xanthine and then to uric acid. Inhibition of IMPDH can therefore lead to a high concentration of uric acid in the blood, which may cause medical problems, including kidney stones, gout, and diabetes. Consequently, the therapeutic use of IMPDH inhibitors is usually accompanied by administration of an inhibitor of XO, such as allopurinol, to prevent accumulation of uric acid in the body.

Humans have two IMPDH isozymes that have similar enzymatic characteristics. The differential roles of the two isozymes are not well understood.

Given the role of IMPDH in synthesis of guanine nucleotides, metabolites in guanine nucleotide synthesis pathways are useful indicators for engagement of IMPDH inhibitors with their target. For example, inhibition of IMPDH prevents de novo synthesis of GTP, so decreased GTP levels are indicative of the degree of IMPDH inhibition in cells capable of de novo GTP synthesis. In addition, IMPDH inhibition results in the accumulation of the substrate IMP, which is then converted to hypoxanthine. As indicated above, conversion of hypoxanthine to uric acid can be blocked by providing inhibitors of XO. Therefore, in subjects that have received inhibitors of both IMPDH and XO, increased levels of hypoxanthine are indicative of the degree of IMPDH inhibition.

The methods of the invention are applicable for therapeutic agents that regulate the activity of other metabolic pathways as well. Examples of such pathways include the anandamide degradation pathway, including the enzyme fatty acid amide hydrolase, which may be targeted by a variety of inhibitors and activators; glycolysis, the citric acid cycle, and the balance between the two, which may targeted by various drug candidates; the pentose phosphate pathway; and the beta-oxidation pathway.

### Therapeutic Agents

In methods of the invention, the dosing regimen may include a therapeutic agent that targets a metabolic pathway, such as one of those described above. The following are examples of therapeutic agents that may be used in methods of the invention.

The therapeutic agent may be an inhibitor of DHODH. Known inhibitors if DHODH include brequinar, leflunomide, and teriflunomide.

Brequinar, 6-fluoro-2-(2′-fluoro-1,1′biphenyl-4-yl)-3-methyl-4-quinoline carboxylic acid, has the following structure:

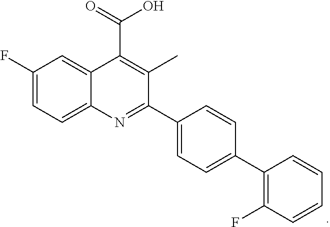 [SMILES: Cc1c(-c2ccc(-c3ccccc3F)cc2)nc2ccc(F)cc2c1C(=O)O]

Brequinar and related compounds are described in, for example, U.S. Pat. Nos. 4,680,299 and 5,523,408, the contents of which are incorporated herein by reference. The use of brequinar to treat leukemia is described in, for example, U.S. Pat. No. 5,032,597 and International Publication No. WO 2017/037022, the contents of which are incorporated herein by reference.

Leflunomide, N-(4′-trifluoromethylphenyl)-5-methylisoxazole-4-carboxamide (I), is described in, for example, U.S. Pat. No. 4,284,786, the contents of which are incorporated herein by reference.

Teriflunomide, 2-cyano-3-hydroxy-N-[4-(trifluoromethyl)phenyl]-2-butenamide, is described in, for example, U.S. Pat. No. 5,679,709, the contents of which are incorporated herein by reference.

The DHODH inhibitor, such as any of the aforementioned compounds, may be provided as an analog, derivative, prodrug, micellar formulation, sustained release formulation, or salt of the pharmacologically active compound.

The therapeutic agent may be an inhibitor of aspartate carbamoyltransferase (also known as aspartate transcarbamoylase or ATCase), such as N-phosphoacetyl-L-aspartate (PALA).

The therapeutic agent may be an inhibitor of orotidine monophosphate decarboxylase (OMPD), such as pyrazofurin.

The therapeutic agent may be an inhibitor of IMPDH. IMPDH inhibitors are known in the art and described in, for example, Cuny, G. D., et al., Inosine-5′-monophosphate dehydrogenase (IMPDH) inhibitors: a patent and scientific literature review (2002-2016), Expert Opin Ther Pat., 2017, June; 27(6):677-690. doi:10.1080/13543776.2017.1280463, the contents of which are incorporated herein by reference.

In some embodiments, the IMPDH inhibitor is tiazofurin. Tiazofurin, which has the systematic name 2-β-D-ribofuranosylthiazole-4-carboxamide, has the following structure:

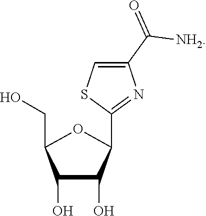

Tiazofurin and methods for making it are known in the art and described in, for example, U.S. Pat. Nos. 4,451,648 and 6,613,896, the contents of which are incorporated herein by reference. Tiazofurin analogs, their activity against tumors, and methods of making them are described in, for example, Popsavin, et al., Synthesis and antiproliferative activity of two new tiazofurin analogues with 2′-amido functionalities, Bioorg. Med. Chem. Lett. 16 (2006) 2773-2776, doi: 10.1016/j.bmcl.2006.02.001; and Popsavin, et al., Synthesis and in vitro antitumor activity of tiazofurin analogues with nitrogen functionalities at the C-2′ position, European Journal of Medicinal Chemistry 111 (2016) 114e125. doi: 10.1016/j.ejmech.2016.01.037, the contents of each of which are incorporated herein by reference.

Tiazofurin is converted inside cells to thiazole-4-carboxamide adenine dinucleotide (TAD). TAD, an analog of NAD⁺, is the form of the compound that interacts with IMDH to block its activity.

Another IMPDH inhibitor is the non-reversible inhibitor mycophenolic acid. Mycophenolic acid has the following structure:

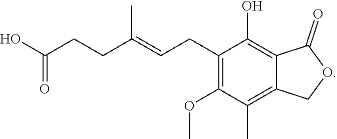 [SMILES: COc1c(C)c2c(c(O)c1C/C=C(\C)CCC(=O)O)C(=O)OC2]

Uses, side effects, and the mechanism of action of mycophenolate are known in the art and described in, for example, Kitchin, J. E., et al., (1997) “Rediscovering mycophenolic acid: A review of its mechanism, side effects, and potential uses” Journal of the American Academy of Dermatology. 37 (3): 445-449. doi:10.1016/S0190-9622(97)70147-6; and Pharmacology North American Edition. Lippincott Williams & Wilkins. 2014, p. 625, ISBN 978-1-4511-9177-6, the contents of each of which are incorporated herein by reference. Salts, derivatives, analogs, and prodrugs of mycophenolic acid are known in the art and described in, for example, Mele T. S., and Halloran, P. F., The use of mycophenolate mofetil in transplant recipients. Immunopharmacology. 2000; 47:215-245; International Publication No. WO 2004/096287A2; and U.S. Pat. No. 7,427,636, the contents of each of which are incorporated herein by reference.

Many other inhibitors of IMPDH are known in the art. For example and without limitation, other IMPDH inhibitors include AS2643361, EICAR, FF-10501, mizoribine, ribavirin, selenazofurin, SM-108, taribavirin, VX-148, VX-497, and VX-944. For example and without limitation, other IMPDH inhibitors include ribavirin, mizoribine, selenazofurin, and taribavirin. Other IMPDH inhibitors are described in Gebeyehu, G., et al., Ribavirin, Tiazofurin, and Selenazofurin: Mononucleotides and Nicotinamide Adenine Dinucleotide Analogues. Synthesis, Structure, and Interactions with IMP Dehydrogenase, J. Med. Chem. 1985, 28, 99-105; and U.S. Pat. Nos. 5,807,876; 6,344,465; 6,395,763; 6,399,773; 6,420,403; 6,518,291; 6,541,496; 6,617,323; 6,624,184; 6,653,309; 6,825,224; 6,867,299; 6,919,335; 6,967,214; 7,053,111; 7,060,720; 7,087,642; 7,205,324; 7,329,681; 7,432,290; 7,777,069; and 7,989,498, the contents of each of which are incorporated herein by reference. Some IMPDH inhibitors have additional modes of action, such as ribavirin, which is also a proposed inhibitor of eukaryotic translation initiation factor 4E (eIF4E).

The therapeutic agent may be an inhibitor of HGPRT. HGPRT inhibitors include 6-mercaptopurine 1,3-dinitroadamantane, acyclovir, and pentamidine.

The therapeutic agent may be an inhibitor of dihydrofolate reductase (DHFR), which reduces dihydrofolic acid to tetrahydrofolic acid in the purine salvage pathway. DHFR inhibitors include aminopterin, methotrexate, pemetrexed, pralatrexate, raltitrexed, and trimetrexate.

The therapeutic agent may be an inhibitor of Bcl-2. Bcl-2 inhibitors include ABT-737, navitoclax, oblimersen, and venetoclax.

The therapeutic agent may be an agent that targets a cell surface marker. For example, the agent may target a cluster of differentiation (CD) marker, such as CD19, CD20, CD22, CD33, or CD52. CD19-targeting agents include blinatumomab. CD20-targeting agents include obinutuzumab, ofatumumab, and rituximab. CD22-targeting agents include inotuzumab ozogamicin, and moxetumomab pasudotox. CD33-targeting agents include 161533 TriKE fusion protein, ²²⁵Ac-lintuzumab, AMG 330, AMG 673, AMV564, BI 836858, gemtuzumab ozogamicin, IMGN779, and vadastuximab talirine (SGN-CD33A). CD52-targeting agents include alemtuzumab.

The therapeutic agent may be an inhibitor of a cytokine receptor. Cytokine receptor inhibitors include anakinra, basiliximab, and daclizumab.

The therapeutic agent may be an agent that interferes with DNA replication. The agent may interfere with DNA replication by inhibiting DNA methylation, inhibiting DNA polymerase, alkylating bases in DNA, direct incorporation into DNA, inhibiting topoisomerase, or any combination of such mechanisms. DNA methylation inhibitors include azacitidine and decitabine. DNA polymerase inhibitors include cladribine, clofarabine, cytarabine, decitabine, deoxyadenosine, deoxycytidine, fludarabine, gemcitabine, nelarabine, pentostatin, and tezacitibine. DNA alkylating agents include bendamustine, busulfan, chlorambucil, cyclophosphamide, and mechlorethamine. Nucleoside analogs or nucleobase analogs that become incorporated into DNA include azacitidine, cladribine, and thioguanine. Topoisomerase inhibitors include amsacrine, aurintricarboxylic acid, camptothecin, daunorubicin, doxorubicin. EGCG, ellipticine, etoposide (VP-16), genistein, HU-331, idarubicin, irinotecan, lamellarin D, mitoxantrone, quercetin, resveratrol, teniposide, and topotecan.

The therapeutic agent may be an inhibitor of a Hedgehog signaling pathway. Inhibitors of Hedgehog signaling pathways include cyclopamine, GANT61, glasdegib, LDE225 (sonidegib/erismodegib), and vismodegib (GDC-0449).

The therapeutic agent may be a tyrosine kinase inhibitor. Tyrosine kinase inhibitors include AC220, bosutinib, dasatinib, gilteritinib, KW-2449, ibrutinib, imatinib, lestaurtinib (CEP-701), midostaurin (PKC412), nilotinib, ponatinib, sorafenib, sunitinib, and tandutinib (MLN518).

The therapeutic agent may be a PI3 kinase inhibitor, such as duvelisib or idelalisib.

The therapeutic agent may be an eIF4A inhibitor, such as eFT226, elatol, hippuristanol, pateamine A, rocaglamide, or silvestrol.

The therapeutic agent may be an asparaginase.

The therapeutic agent may be hydroxyurea, omacetaxine, prednisone, or vincristine.

The therapeutic agent, such as any of the aforementioned agents, may be provided as an analog, derivative, prodrug, micellar formulation, sustained release formulation, or salt of the pharmacologically active compound.

### Measuring Levels of a Drug or Biomarker

Methods of the invention include analysis of measured levels of a metabolite, such as DHO, and a therapeutic agent, such as brequinar, in samples from a subject. The methods may include measurement of the levels of the metabolite and therapeutic agent.

In some embodiments, the metabolite or therapeutic agent is measured by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi:10.1002/elps.200500398; Kolch et al., Capillary electrophoresis-mass spectrometry as a powerful tool in clinical diagnosis and biomarker discovery, Mass Spectrom Rev. 24 (6): 959-77. (2005) doi:10.1002/mas.20051; Kanu et al., Ion mobility-mass spectrometry, Journal of Mass Spectrometry, 43 (1): 1-22 (2008) doi:10.1002/jms.1383, the contents of which are incorporated herein by reference.

A sample may be obtained from any organ or tissue in the individual to be tested, provided that the sample is obtained in a liquid form or can be pre-treated to take a liquid form. For example and without limitation, the sample may be a blood sample, a urine sample, a serum sample, a semen sample, a sputum sample, a lymphatic fluid sample, a cerebrospinal fluid sample, a plasma sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, a phlegm sample, a saliva sample, a sweat sample, or a combination of such samples. The sample may also be a solid or semi-solid sample, such as a tissue sample, feces sample, or stool sample, that has been treated to take a liquid form by, for example, homogenization, sonication, pipette trituration, cell lysis etc. For the methods described herein, it is preferred that a sample is from plasma, serum, whole blood, or sputum.

The sample may be kept in a temperature-controlled environment to preserve the stability of the metabolite. For example, DHO is more stable at lower temperatures, and the increased stability facilitates analysis of this metabolite from samples. Thus, samples may be stored at, or 4° C., −20° C., or −80° C.

In some embodiments, a sample is treated to remove cells or other biological particulates. Methods for removing cells from a blood or other sample are well known in the art and may include e.g., centrifugation, sedimentation, ultrafiltration, immune selection, etc.

The subject may be an animal (such as a mammal, such as a human). The subject may be a pediatric, a newborn, a neonate, an infant, a child, an adolescent, a pre-teen, a teenager, an adult, or an elderly patient. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The sample may be obtained from an individual before or after administration to the subject of a DHODH inhibitor. For example, the sample may be obtained 1 hour, 2 hours, 4 hours, 6 hours, 8 hours, 12 hours, 24 hours, 36 hours, 2 days, 3 days, 4 days, 5 days, 6 days, 7 days or more before administration of a DHODH inhibitor, or it may be obtained 1 hour, 2 hours, 4 hours, 6 hours, 8 hours, 12 hours, 24 hours, 36 hours, 2 days, 3 days, 4 days, 5 days, 6 days, 7 days or more after administration of a DHODH inhibitor.

### Providing Doses of a Therapeutic Agent

Methods of the invention may include providing a DHODH inhibitor or other therapeutic agent to a subject according to an adjusted dosing regimen, as described above. Alternatively or additionally, methods may include instructions for providing a DHODH inhibitor or other therapeutic agent to a subject according to an adjusted dosing regimen. Providing the DHODH inhibitor or other therapeutic agent to the subject may include administering it to the subject. A dose may be administered as a single unit or in multiple units. The DHODH inhibitor or other therapeutic agent may be administered by any suitable means. For example and without limitation, the DHODH inhibitor or other therapeutic agent may be administered orally, intravenously, enterally, parenterally, dermally, buccally, topically, transdermally, by injection, intravenously, subcutaneously, nasally, pulmonarily, or with or on an implantable medical device (e.g., stent or drug-eluting stent or balloon equivalents).

### Disorders

The methods of the invention are useful for determining adjustments to drug dosing regimens to treat or prevent a disorder. The disorder may be a disease, disorder, or condition for which administration of a DHODH inhibitor provides a therapeutic benefit.

For example and without limitation, one disorder that can be treated by methods of the invention is acute myeloid leukemia (AML). In AML, myeloblasts arrested in an early stage of differentiation proliferate in an uncontrolled manner and interfere with the development of other blood cells in the bone marrow. Inhibitors of dihydroorotate dehydrogenase (DHODH), an enzyme involved in pyrimidine synthesis, cause differentiation of myeloblasts and prevent their leukemia-initiating activity. The role of DHODH in AML is described in Sykes et al., Inhibition of Dihydroorotate Dehydrogenase Overcomes Differentiation Blockade in Acute Myeloid Leukemia, Cell 167, 171-186, Sep. 22, 2016; dx.doi.org/10.1016/j.cell.2016.08.057, the contents of which are incorporate herein by reference.

The use of DHODH inhibitors to treat AML requires a precise dosing regimen. Care must be taken to avoid excessive inhibition of DHODH. DHODH is an essential enzyme, and homozygous recessive mutations in DHODH cause Miller syndrome, a disorder characterized by multi-organ dysfunction. In a mouse model of AML, daily administration of high doses of the DHODH inhibitor brequinar lead to weight loss, anemia, and thrombocytopenia. At the same time, sustained exposure to brequinar is necessary to inhibit DHODH for sufficient periods to produce a therapeutic effect in the mouse AML model. Without wishing to be bound by theory, one hypothesis for the narrow therapeutic window of brequinar in treating AML in both the mouse model and in humans is that malignant cells display an increased sensitivity to DHODH inhibition. In particular, normal cells may be able to tolerate periods of nucleotide starvation that kill cancer cells due to the elevated metabolic needs of the latter.

The narrow therapeutic window of DHODH inhibition likely applies to other disorders as well. For example, brequinar was evaluated for treatment of solid tumor malignancies and found to be ineffective when administered over a 5-day period followed by a 3-week gap or once per week for three weeks followed by a 1-week gap. See Arteaga, C. L. et al. (1989) Phase I clinical and pharmacokinetic trial of Brequinar sodium (DuP 785; NSC 368390) Cancer Res. 49, 4648-4653; Burris, H. A., et al. (1998) Pharmacokinetic and phase I studies of brequinar (DUP 785; NSC 368390) in combination with cisplatin in patients with advanced malignancies, Invest. New Drugs 16, 19-27; Noe, D. A., et al. (1990) Phase I and pharmacokinetic study of brequinar sodium (NSC 368390), Cancer Res. 50, 4595-4599; Schwartsmann, G. et al. (1990) Phase I study of Brequinar sodium (NSC 368390) in patients with solid malignancies, Cancer Chemother. Pharmacol. 25, 345-351, the contents of each of which are incorporated herein by reference. However, brequinar may be effective for treatment of other cancers if the drug is administered in a manner that provides sustained DHODH inhibition.

It is understood that the aforementioned examples are provided for illustrative purposes only and that the methods of the invention can be used for treatment of any disorder or disease in which the measured level of DHO can be used to assess target engagement. The disorder may be one in which inhibiting DHODH is of therapeutic benefit. The disorder may be cancer. The cancer may include a solid tumor or hematological tumor. The cancer may be acute lymphoblastic leukemia (ALL), adult T cell leukemia/lymphoma (ATLL), bladder cancer, breast cancer, such as triple negative breast cancer (TNBC), glioma, head and neck cancer, leukemia, such as AML, lung cancer, such as small cell lung cancer and non-small cell lung cancer, lymphoma, multiple myeloma, neuroblastoma, osteosarcoma, ovarian cancer, prostate cancer, or renal cell cancer. The disorder may have a genetic mutation such as MYC amplification or PTEN loss that leads to increased dependence on the metabolic pathway, such as increased “addiction” to glutamine. The disorder may be an inflammatory or autoimmune disorder, such as arthritis, hepatitis, chronic obstructive pulmonary disease, multiple sclerosis, or tendonitis. The disorder may be a psychiatric disorder, such as anxiety, stress, obsessive-compulsive disorder, depression, panic disorder, psychosis, addiction, alcoholism, attention deficit hyperactivity, agoraphobia, schizophrenia, or social phobia. The disorder may be a neurological or pain disorder, such as epilepsy, stroke, insomnia, dyskinesia, peripheral neuropathic pain, chronic nociceptive pain, phantom pain, deafferentation pain, inflammatory pain, joint pain, wound pain, post-surgical pain, or recurrent headache pain, appetite disorders, or motor activity disorders. The disorder may be a neurodegenerative disorder, such as Alzheimer's disease, Parkinson's disease, or Huntington's disease.

The disorder may include a class or subset of patients having a disease, disorder, or condition. For example, AML cases are classified based on cytological, genetic, and other criteria, and AML treatment strategies vary depending on classification. One AML classification system is provided by the World Health Organization (WHO). The WHO classification system includes subtypes of AML provided in Table 1 and is described in Falini B, et al. (October 2010) “New classification of acute myeloid leukemia and precursor-related neoplasms: changes and unsolved issues” Discov Med. 10 (53): 281-92, PMID 21034669, the contents of which are incorporated herein by reference.

TABLE 1

| Name | Description |
|---|---|
| Acute myeloid | Includes: |
| leukemia with | AML with translocations between chromosome 8 and 21 - |
| recurrent | [t(8;21)(q22;q22);] RUNX1/RUNX1T1; (ICD-O 9896/3); |
| genetic | AML with inversions in chromosome 16 - [inv(16)(p13.1 q22)] or internal |
| abnormalities | translocations in it - [t(16;16)(p13.1;q22);] CBFB/MYH11; (ICD-O |
| | 9871/3); |
| | Acute promyelocytic leukemia with translocations between chromosome |
| | 15 and 17 - [t(15;17)(q22;q12);] RARA/PML; (ICD-O 9866/3); |
| | AML with translocations between chromosome 9 and 11 - |
| | [t(9;11)(p22;q23);] MLLT3/MLL; |
| | AML with translocations between chromosome 6 and 9 - |
| | [t(6;9)(p23;q34);] DEK/NUP214; |
| | AML with inversions in chromosome 3 - [inv(3)(q21q26.2)] or internal |
| | translocations in it - [t(3;3)(q21;q26.2);] RPN1/EVI1; |
| | Megakaryoblastic AML with translocations between chromosome |
| | 1 and 22 - [t(1;22)(p13;q13);] RBM15/MKL1; |
| | AML with mutated NPM1 |
| | AML with mutated CEBPA |
| AML with | Includes people who have had a prior documented myelodysplastic |
| myelodysplasia- | syndrome (MDS) or myeloproliferative disease (MPD) that then has |
| related changes | transformed into AML, or who have cytogenetic abnormalities characteristic |
| | for this type of AML (with previous history of MDS or MPD that has gone |
| | unnoticed in the past, but the cytogenetics is still suggestive of MDS/MPD |
| | history). This category of AML occurs most often in elderly people and often |
| | has a worse prognosis. Includes: |

AML with complex karyotype
Unbalanced abnormalities

AML with deletions of chromosome 7 - [del(7q);]
AML with deletions of chromosome 5 - [del(5q);]
AML with unbalanced chromosomal aberrations in chromosome 17 -
[i(17q)/t(17p);]
AML with deletions of chromosome 13 - [del(13q);]
AML with deletions of chromosome 11 - [del(11q);]
AML with unbalanced chromosomal aberrations in chromosome 12 -
[del(12p)/t(12p);]
AML with deletions of chromosome 9 - [del(9q);]
AML with aberrations in chromosome X - [idic(X)(q13);]

Balanced abnormalities

AML with translocations between chromosome 11 and 16 -
[t(11;16)(q23;q13.3);], unrelated to previous chemotherapy or
ionizing radiation
AML with translocations between chromosome 3 and 21 -
[t(3;21)(q26.2;q22.1);], unrelated to previous chemotherapy or
ionizing radiation
AML with translocations between chromosome 1 and 3 -
[t(1;3)(p36.3;q21.1);]
AML with translocations between chromosome 2 and 11 -
[t(2;11)(p21;q23);], unrelated to previous chemotherapy or ionizing
radiation
AML with translocations between chromosome 5 and 12 -
[t(5;12)(q33;p12);]
AML with translocations between chromosome 5 and 7 -
[t(5;7)(q33;q11.2);]
AML with translocations between chromosome 5 and 17 -
[t(5;17)(q33;p13);]
AML with translocations between chromosome 5 and 10 -
[t(5;10)(q33;q21);]
AML with translocations between chromosome 3 and 5 -
[t(3;5)(q25;q34);]

| | |
|---|---|
| Therapy-related | Includes people who have had prior chemotherapy and/or radiation and |
| myeloid | subsequently develop AML or MDS. These leukemias may be characterized |
| neoplasms | by specific chromosomal abnormalities, and often carry a worse prognosis. |
| Myeloid | Includes myeloid sarcoma. |
| sarcoma | |
| Myeloid | Includes so-called ″transient abnormal myelopoiesis″ and ″Myeloid leukemia |
| proliferations | associated with Down syndrome″ |
| related to Down | |
| syndrome | |
| Blastic | Includes so-called ″blastic plasmacytoid dendritic cell neoplasm″ |
| plasmacytoid | |
| dendritic cell | |
| neoplasm | |
| AML not | Includes subtypes of AML that do not fall into the above categories |
| otherwise | AML with minimal differentiation |
| categorized | AML without maturation |
| | AML with maturation |
| | Acute myelomonocytic leukemia |
| | Acute monoblastic and monocytic leukemia |
| | Acute erythroid leukemia |
| | Acute megakaryoblastic leukemia |
| | Acute basophilic leukemia |
| | Acute panmyelosis with myelofibrosis |

An alternative classification scheme for AML is the French-American-British (FAB) classification system. The FAB classification system includes the subtypes of AML provided in Table 2 and is described in Bennett J M, et al. (August 1976). “Proposals for the classification of the acute leukemias. French-American-British (FAB) co-operative group” Br. J. Haematol. 33 (4): 451-8, doi:10.1111/j.1365-2141.1976.tb03563.x. PMID 188440; and Bennett J M, et al. (June 1989) “Proposals for the classification of chronic (mature) B and T lymphoid leukemias. French-American-British (FAB) Cooperative Group” J. Clin. Pathol. 42 (6): 567-84, doi:10.1136/jcp.42.6.567, PMC 1141984, PMID 2738163, the contents of each of which are incorporated herein by reference.

TABLE 2

| Type | Name | Cytogenetics |
|---|---|---|
| M0 | acute myeloblastic leukemia, minimally | |
| | differentiated | |
| M1 | acute myeloblastic leukemia, without maturation | |
| M2 | acute myeloblastic leukemia, with granulocytic | t(8;21)(q22;q22), t(6;9) |
| | maturation | |
| M3 | promyelocytic, or acute promyelocytic | t(15;17) |
| | leukemia (APL) | |
| M4 | acute myelomonocytic leukemia | inv(16)(p13q22), del(16q) |
| M4eo | myelomonocytic together with bone | inv(16), t(16; 16) |
| | marrow eosinophilia | |
| M5 | acute monoblastic leukemia (M5a) or acute | del (11q), t(9;11), t(11;19) |
| | monocytic leukemia (M5b) | |
| M6 | acute erythroid leukemias, including | |
| | erythroleukemia (M6a) and very rare pure | |
| | erythroid leukemia (M6b) | |
| M7 | acute megakaryoblastic leukemia | t(1;22) |

The disorder may include a sub-population of patients. For example, the patients may be pediatric, newborn, neonates, infants, children, adolescent, pre-teens, teenagers, adults, or elderly. The patients may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

### Safety and Adverse Events

Methods of the invention may include determining whether a subject suffers from an adverse event while receiving one or more therapeutic agents. Determining whether a subject suffers from an adverse event may include determining whether the adverse is unsafe, whether it is caused by a therapeutic agent, and/or its level of severity. Suitable measures for characterizing adverse events are known in the art and described in, for example, the revised NCI Common Terminology Criteria for Adverse Events (CTCAE) version 4.03, available at ctep.cancer.gov/protocolDevelopment/electronic_applications/ctc.htm, the contents of which are incorporated herein by reference.

A therapeutic agent may be deemed safe and/or tolerable if it does not cause adverse events that meet defined criteria. Safety and/or tolerability may be assessed by looking for adverse events over a defined period when the subject receives the therapeutic agent. For example, the period may be about 1 week, 2 weeks, 3 weeks, 4 weeks, 5 weeks, 6 weeks, 7 weeks, 8 weeks, 2 months, 3 months, 4 months, 5 months, or 6 months.

For example and without limitation, administration of a therapeutic agent, such as brequinar, according to a dosing schedule may be deemed safe and/or tolerable if the subject experiences no ≥Grade 3, treatment-emergent, study-drug-related, non-hematologic adverse events over a period. The period may be 42 days. Safety and/or tolerability assessment may include certain exceptions to the non-hematologic Grade 3 criteria, such as one or more of those provided in Table 3.

TABLE 3

| Condition | Exception Description |
|---|---|
| Nausea/ | Grade 3 nausea and/or emesis resolving to less than or equal to Grade 2 or |
| Emesis | baseline within 1 week after the use of an optimal antiemetic regimen |
| | based on standard practice. |
| Diarrhea | Grade 3 diarrhea resolving to less than or equal to Grade 1 or baseline |
| | within 1 week after receiving the maximal supportive therapy based on |
| | standard practice. |
| Laboratory | Asymptomatic Grade 3 laboratory abnormalities that are not considered to |
| abnormalities | be clinically significant. |
| Mucositis | Grade 3 with duration < 1 week |
| Fatigue | Grade 3 with duration < 2 weeks |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method for adjusting a dose and/or schedule of dosing of an agent provided to a subject that has a disorder, the method comprising:
receiving a sample from a subject that has received an initial dose of an agent;
conducting a first assay on the sample to assess whether a plasma level of the agent is within a therapeutic window;
conducting a second assay on the sample that assesses a biomarker that is indicative of whether the agent has engaged a biological target, wherein the assay assesses whether the biomarker is below or above a threshold level; and
generating a report that provides (i) a plasma level of the agent with respect to the therapeutic window and (ii) a level of the biomarker with respect to the threshold level; wherein the report provides guidance to a doctor on how to determine a dosage adjustment for a subsequent dose of the agent and/or an adjustment of a schedule of dosing of the agent to be provided to the subject based on (i) and (ii) of the report.

2. The method of claim 1, wherein:
(i) the dosage adjustment is at least one selected from the group consisting of: increase the dosage by a certain amount, decrease the dosage by a certain amount, and make no adjustment to the dosage; and
(ii) the adjustment of the schedule of dosing is at least one selected from the group consisting of: increase a frequency in which the agent is provided; and decrease the frequency in which the agent is provided;
wherein the method comprises implementing (i) alone, (ii) alone, or any combination of (i) and (ii).

3. The method of claim 1, wherein the agent inhibits an enzyme in a metabolic pathway.

4. The method of claim 3, wherein the metabolite is a substrate of the enzyme.

5. The method of claim 4, wherein the metabolic pathway is a nucleotide synthesis pathway.

6. The method of claim 5, wherein the enzyme is selected from the group consisting of aspartate transcarbamoylase, dihydrooratase, dihydroorotate dehydrogenase, orotidine 5′-monophosphate (OMP) decarboxylase, inositol monophosphate dehydrogenase, and orotate phosphoribosyl transferase.

7. The method of claim 6, wherein the metabolite is selected from the group consisting of N-carbamoylaspartate, dihydroorotate, orotate, orotidine 5′-monophosphate (OMP), inosine monophosphate (IMP), uridine, and uridine monophosphate (UMP).

8. The method of claim 7, wherein the agent comprises one selected from the group consisting of PALA (N-phosphoacetyl-L-aspartate), pyrazofurin, brequinar, a brequinar analog, a brequinar derivative, a brequinar prodrug, a micellular formulation of brequinar, a brequinar salt, mizoribine, mycophenolic acid, ribavirin, selenazofurin, taribavirin, and tiazofurin.

9. The method of claim 1, wherein the disorder is cancer.

10. The method of claim 1, further comprising: providing the agent to the subject at the determined adjusted dose and/or adjusted schedule.

11. A method for adjusting a dose and/or dosing schedule of an agent to be provided to a subject that has a disorder and that has already received an initial dose of the agent, the method comprising:
receiving, after a subject has received an initial dose of an agent, a report that provides: (i) a plasma level of the agent with respect to a therapeutic window; and (ii) a level of a biomarker with respect to a threshold level, wherein the biomarker is indicative of whether an agent has engaged a biological target;
determining, based on (i) and (ii) in the report, a dosage adjustment for a subsequent dose of the agent to be provided to the subject and/or an adjustment of a schedule of dosing of the agent to be provided to the subject; and
providing the agent to the subject at the determined adjusted dose and/or adjusted dosing schedule.

12. The method of claim 11, wherein:
(i) the dosage adjustment is at least one selected from the group consisting of: increase the dosage by a certain amount, decrease the dosage by a certain amount, and make no adjustment to the dosage; and
(ii) the adjustment of the schedule of dosing is at least one selected from the group consisting of: increase a frequency in which the agent is provided; and decrease the frequency in which the agent is provided;
wherein the method comprises implementing (i) alone, (ii) alone, or any combination of (i) and (ii).

13. The method of claim 12, wherein the agent inhibits an enzyme in a metabolic pathway.

14. The method of claim 13, wherein the metabolite is a substrate of the enzyme.

15. The method of claim 14, wherein the metabolic pathway is a nucleotide synthesis pathway.

16. The method of claim 15, wherein the enzyme is selected from the group consisting of aspartate transcarbamoylase, dihydrooratase, dihydroorotate dehydrogenase, orotidine 5′-monophosphate (OMP) decarboxylase, inositol monophosphate dehydrogenase, and orotate phosphoribosyl transferase.

17. The method of claim 16, wherein the metabolite is selected from the group consisting of N-carbamoylaspartate, dihydroorotate, orotate, orotidine 5′-monophosphate (OMP), inosine monophosphate (IMP), uridine, and uridine monophosphate (UMP).

18. The method of claim 17, wherein the agent comprises one selected from the group consisting of PALA (N-phosphoacetyl-L-aspartate), pyrazofurin, brequinar, a brequinar analog, a brequinar derivative, a brequinar prodrug, a micellular formulation of brequinar, a brequinar salt, mizoribine, mycophenolic acid, ribavirin, selenazofurin, taribavirin, and tiazofurin.

19. The method of claim 11, wherein the disorder is cancer.

20. The method of claim 11, further comprising: providing the agent to the subject at the determined adjusted dose and/or adjusted schedule.
